(19) United States
(12) Patent Application Publication
(10) Pub. No.: US 2020/0337937 A1
(43) Pub. Date: Oct. 29, 2020

(54) ATAXIA REACTIVE COMPRESSION SLEEVE

(71) Applicants: Joseph McMillan, Maricopa, AZ (US); Hansol Moon, Gilbert, AZ (US); Joshua Pace, Mesa, AZ (US); Arizona Board of Regents on behalf of Arizona State University, Scottsdale, AZ (US); Dignity Health, San Francisco, CA (US)

(72) Inventors: Thomas Sugar, Chandler, AZ (US); Yugesh Kumar Gajendran, Tempe, AZ (US); Joseph McMillan, Maricopa, AZ (US); Hansol Moon, Gilbert, AZ (US); Joshua Pace, Mesa, AZ (US); Saivimal Sridar, Mesa, AZ (US); Pham Huy Nguyen, Mesa, AZ (US); Jeremy Palmiscno, Phoenix, AZ (US); Kristin Palmiscno, Phoenix, AZ (US); Steven Frey, Phoenix, AZ (US); Will Meredith, Phoenix, AZ (US)

(21) Appl. No.: 16/859,501

(22) Filed: Apr. 27, 2020

Related U.S. Application Data
(60) Provisional application No. 62/838,828, filed on Apr. 25, 2019.

(51) Int. Cl. A61H 9/00

(52) U.S. Cl. CPC A61H 9/0078; A61H 2201/1238; A61H 2205/065; A61H 2201/1635; A61B 5/1101; A61H 2201/501; A61H 2201/5084; A61H 2201/5071; A61H 2201/5097; A61H 2201/165

(57) ABSTRACT

A soft wearable medical device may comprise a soft compliant pumpactuator or actuators at least partially disposed in a glove assembly. Responsive to a detection of a tremor, such as an ataxia tremor, an actuator at least partially inflates to provide a stabilizing counterforce and reduce or eliminate the tremor.

### CROSS-REFERENCE TO RELATED APPLICATION

This application is a non-provisional patent application of, and claims priority to, U.S. Provisional Pat. App. No. 62/838,828 filed Apr. 25, 2019 and entitled “Ataxia Reactive Compression Sleeve,” which is incorporated herein by reference in its entirety (except for any subject matter disclaimers or disavowals, and except to the extent of any conflict with the disclosure of the present application, in which case the disclosure of the present application shall control).

### TECHNICAL FIELD

The present disclosure relates soft robotic systems, and in particular to systems for use by individuals afflicted by ataxia.

### BACKGROUND

Many forms of ataxia result in difficulty controlling coarse motor function, often resulting in tremor-like instability within different joints. Ataxia tremors can be stabilized by providing the patient with a small amount of pressure on a stable surface as a reference point. When this reference is provided, the patient gains better stability. The three main forms of ataxia are hereditary ataxia, Cerebellar ataxia (ILOA), and acquired ataxia. Acquired ataxia can develop the following conditions such as a stroke or other brain-related disease that affects motor control. The most common form of inherited ataxia is Friedreich ataxia, which affects 1 in every 50,000 people. Ataxia can lead to a lack of sensory feedback from affected limbs as well as a general lack of control in these areas.

People who suffer from ataxia often have a lower quality of life, as ataxia can make simple motions difficult. Those who deal with ataxia often find basic hand motions extremely difficult and rely on others to help them. Accordingly, systems and methods usable to help those afflicted with ataxia be more independent, and give them control of their hand motion, are desirable.

### SUMMARY

A soft wearable medical device is disclosed herein. In an exemplary embodiment, a compression sleeve wrist tremor stabilization system comprises a first pneunet attached to a glove and disposed at the underside of the wrist, and a second pneunet attached to the glove and disposed over the thenar eminence muscle group.

In another exemplary embodiment, a method of stabilizing an ataxia tremor comprises detecting, via an accelerometer, an ataxia tremor; and inflating, by a compressor, at least one of a first pneunet attached to a glove and disposed at the underside of the wrist or a second pneunet attached to the glove and disposed over the thenar eminence muscle group.

The foregoing features and elements may be combined in various combinations without exclusivity, unless expressly indicated otherwise. These features and elements as well as the operation thereof will become more apparent in light of the following description and the accompanying drawings. It should be understood, however, the following description and drawings are intended to be exemplary in nature and non-limiting. The contents of this section are intended as a simplified introduction to the disclosure, and are not intended to limit the scope of any claim.

### BRIEF DESCRIPTION OF THE DRAWINGS

With reference to the following description and accompanying drawings:

FIG. 1A illustrates an exemplary system in accordance with various exemplary embodiments;

FIG. 1B illustrates an exemplary system in accordance with various exemplary embodiments;

FIG. 1C illustrates an exemplary system in accordance with various exemplary embodiments;

FIG. 2 illustrates an exemplary pneunet in accordance with various exemplary embodiments;

FIG. 3A illustrates an exemplary system in accordance with various exemplary embodiments;

FIG. 3B illustrates an exemplary system in accordance with various exemplary embodiments, showing preservation of range of motion; and

FIG. 3C illustrates an exemplary system in accordance with various exemplary embodiments.

### DETAILED DESCRIPTION

The following description is of various exemplary embodiments only, and is not intended to limit the scope, applicability or configuration of the present disclosure in any way. Rather, the following description is intended to provide a convenient illustration for implementing various embodiments including the best mode. As will become apparent, various changes may be made in the function and arrangement of the elements described in these embodiments without departing from principles of the present disclosure.

For the sake of brevity, conventional techniques and components may not be described in detail herein. Furthermore, the connecting lines shown in various figures contained herein are intended to represent exemplary functional relationships and/or physical couplings between various elements. It should be noted that many alternative or additional functional relationships or physical connections may be present in exemplary systems and/or components thereof.

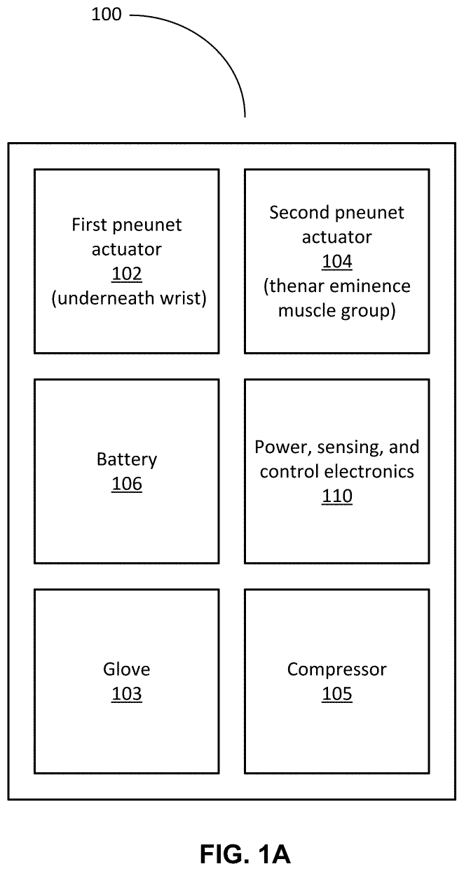

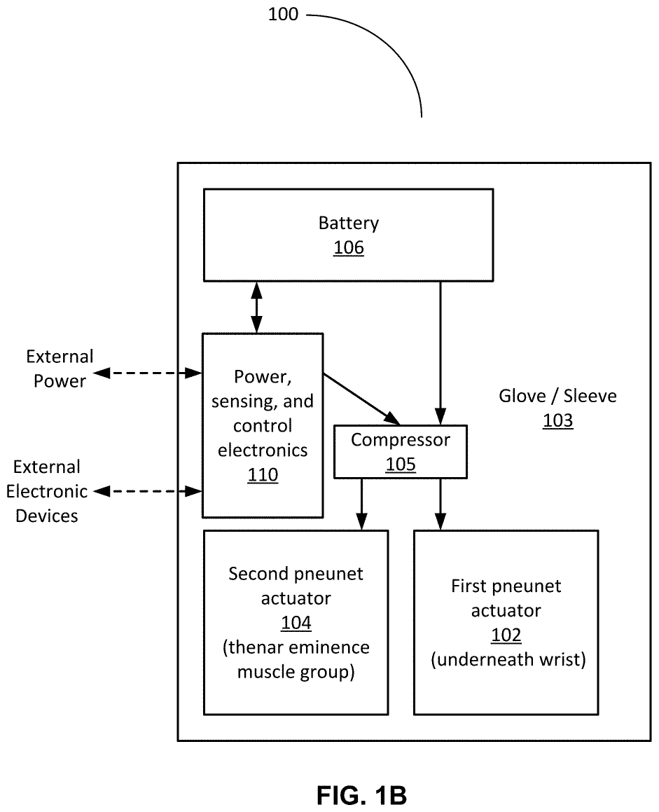

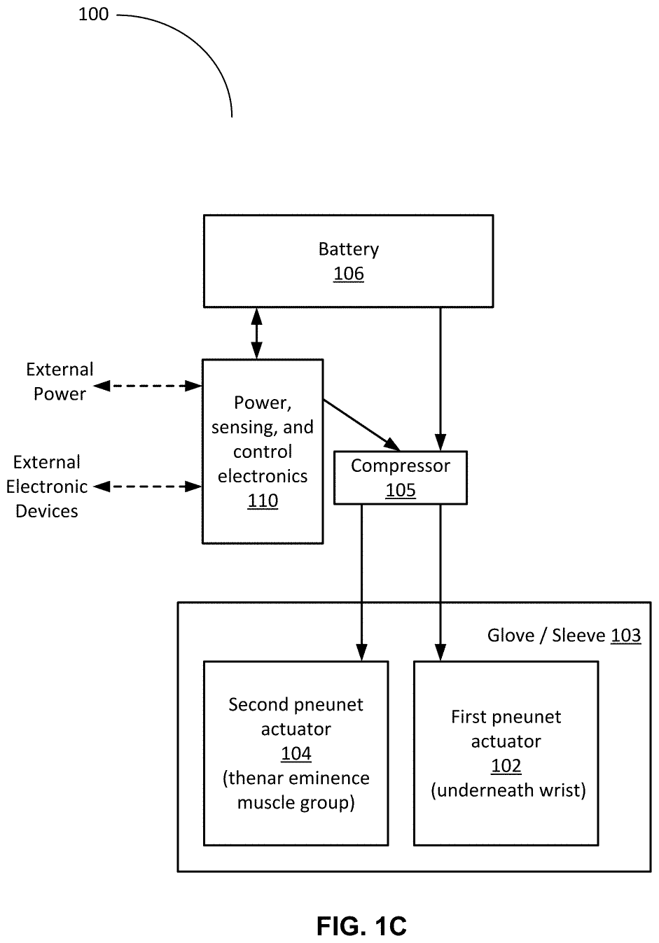

With reference now to FIGS. 1A, 1B, and 1C, in various exemplary embodiments, an ataxia reactive compression sleeve, for example system 100, is configured to stabilize tremors on a patient's wrist, for example when his/her elbow is fully stabilized on a reference point. In various exemplary embodiments, system 100 utilizes a silicone-based soft-robotic actuator. However, any suitable soft robotic actuator may be utilized, as desired. The soft robotic actuator is configured to act as an artificial bounded digit that extends from the wrist to provide a reference point. System 100 is operable to provide a position reference to patients with ataxia to at least partially alleviate wrist tremors caused by the pathology.

An exemplary system 100 comprises a wrist tremor stabilization device configured to allow allow maximum ROM (range of motion) for the user and help them achieve fine motor control. In some embodiments, a soft compliant actuator, such as a pneunet, which can provide a counterforce is utilized for actuation. The soft compliant actuator may comprise a thermoplastic polyurethane material and be covered in fabric. However, any suitable actuator may be utilized, as desired. An exemplary pneunet has a ribbed chamber made out of a suitable material or materials, for example silicone rubber. When the chamber is inflated, due to differences in stiffness between the two sides, the pneunet bends. This bending motion is the actuation used to counter motion due to the tremors.

In one embodiment, system 100 utilizes two soft compliant actuators, a first pneunet 102 and a second pneunet 104. First pneunet 102 is attached to a glove/sleeve 103 and is disposed generally at the underside of the wrist, providing a counter force in that location. Second pneunet 104 is disposed generally on the side over the thenar eminence muscle group. However, it will be appreciated that any suitable number of pneunets may be utilized, for example three, four, five, or even more pneunets, and such pneunets may be disposed in system 100 in such a manner as to be positioned to exert force on any suitable number of muscles or muscle groups. Pneunets 102/104 may be configured to provide any suitable force; moreover, pneunets 102/104 may be low profile, low pressure for safety, convenience, and ease of operation.

In various exemplary embodiments, system 100 comprises a glove/sleeve 103 that provides resistance against ataxia related movements. In various exemplary embodiments, glove/sleeve 103 comprises a stretchable material and/or one or more straps or fasteners in order to secure the device to a user. Due in part to use of these flexible materials, system 100 is quick to don (typically, less than 10 seconds) and quick to doff (again, typically less than 10 seconds).

In operation, system 100 reacts to the user's motion to adjust itself, optimizing the response. A target is to reduce the tremors experienced by those suffering from ataxia by providing a reference point. System 100 provides counter-forces that generate a desired reference point.

In various exemplary embodiments, system 100 is pneumatic, utilizing an air pump 105 or other suitable compressor to provide actuation to pneunet 102 and/or 104. System 100 may be powered by a battery 106 disposed on and/or integrated within system 100; alternatively, system 100 may be powered by a remote battery or mains power and coupled via a power cord. Battery 106 may be a primary battery, a rechargeable battery, and/or the like, and system 100 may integrate electronic components and connections to permit wired and/or wireless charging of battery 106.

In various exemplary embodiments, glove/sleeve 103 may comprise a first component or materials and be coupled to a second component or materials. In other embodiments, glove/sleeve 103 is monolithic and/or formed from the same materials such that the form a single common unit.

As compared to prior approaches: system 100 can be worn by the patient throughout the day; system 100 does not hinder the patient's range of motion of the wrist and usage of palm due to the usage of the soft actuator(s); system 100 response time is significantly shorter than the existing wrist stabilizers as it is built specifically for ataxia application; system 100 offers a flexible and concealable design, for example being slim enough to fit under clothing and/or with the actuators being concealed beneath glove and/or sleeve portions; system 100 does not limit the maximum range of motion of the wrist; system 100 utilizes a battery powered wearable compressor for the actuation; and system 100 offers a modular design that allows the replacement of actuators. Additionally, prior devices and compression sleeves available in the market now are rigid and passive, whereas exemplary embodiments such as system 100 are flexible, and do not hinder the motion of the wrist, giving it a maximum range of motion.

In various exemplary embodiments, system 100 comprises power, sensing, and control electronics such as a control system 110 (comprising one or more microcontrollers, microprocessors, integrated circuits, system-on-chip, and/or the like, for example configured for low power operation and/or to minimize power draw in order to extend battery life). In some exemplary embodiments, control system 110 comprises a 9-degree of freedom inertial measurement unit, pressure transducers to limit forces generated by pneunets 102/104, and/or the like.

Control system 100 is configured to detect ataxia tremors (for example, via one or more accelerometers, pressure sensors, and/or the like disposed on and/or in glove/sleeve 103). Control system 110 may comprise communication components, for example wireless connectivity via Bluetooth, IEEE 802.11, and/or the like, for example in order to provide operational data, patient data, system diagnostics, and/or the like to a remote system; moreover, control system 110 may be configured to receive commands and updates from a remote system, modify operation (for example, changing inflation profiles or triggers for a pneunet responsive to changes in patient conditions). When an ataxia tremor is detected, control system 110 generates commands to a compressor, causing inflation of pneunet 102 and/or 104 in order to generate a stabilizing counterforce to reduce or alleviate the tremor. Pneunet 102 and/or 104 may be deflated, for example responsive to completion of a countdown timer triggered upon inflation, responsive to control system 110 no longer detecting (or detecting the end of) an ataxia tremor, and/or the like.

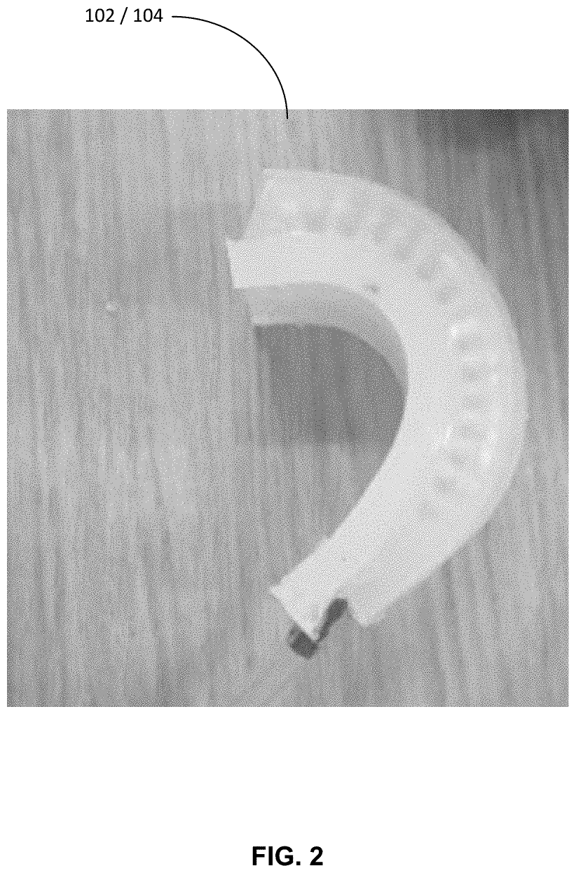

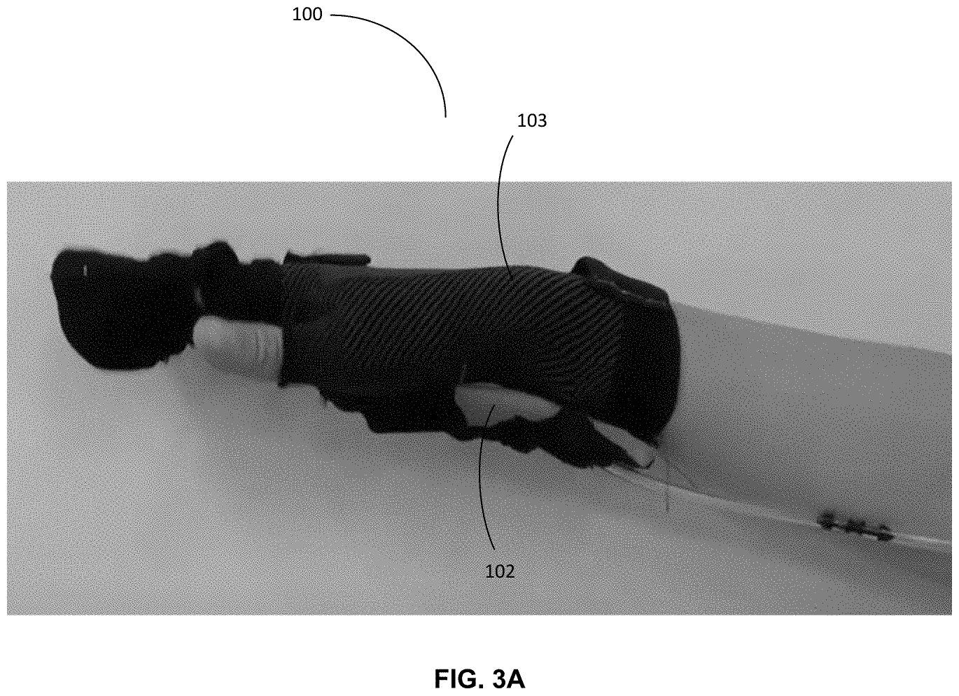

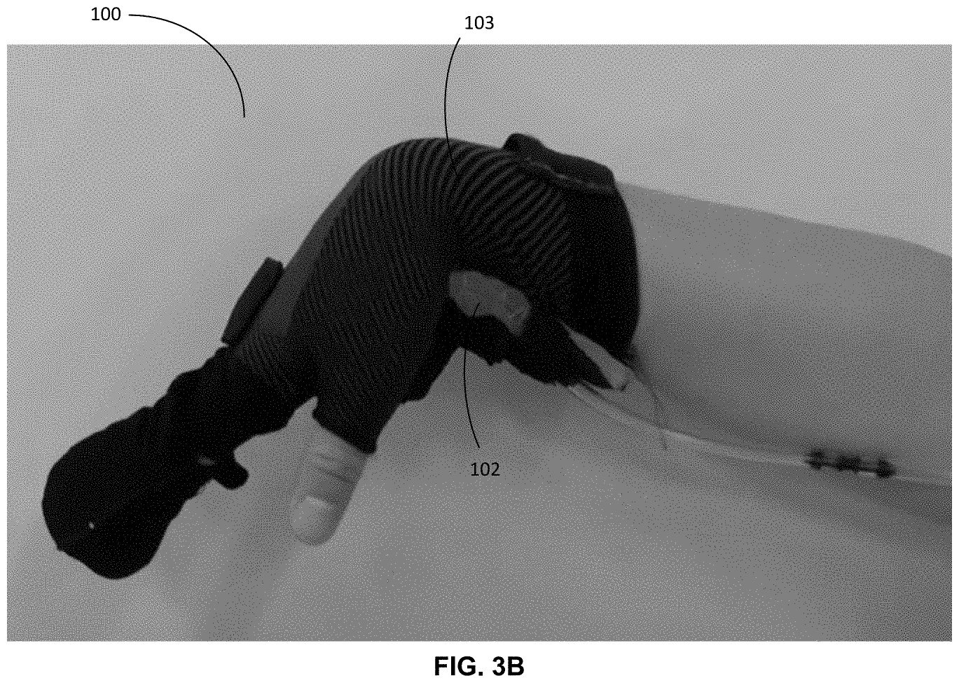

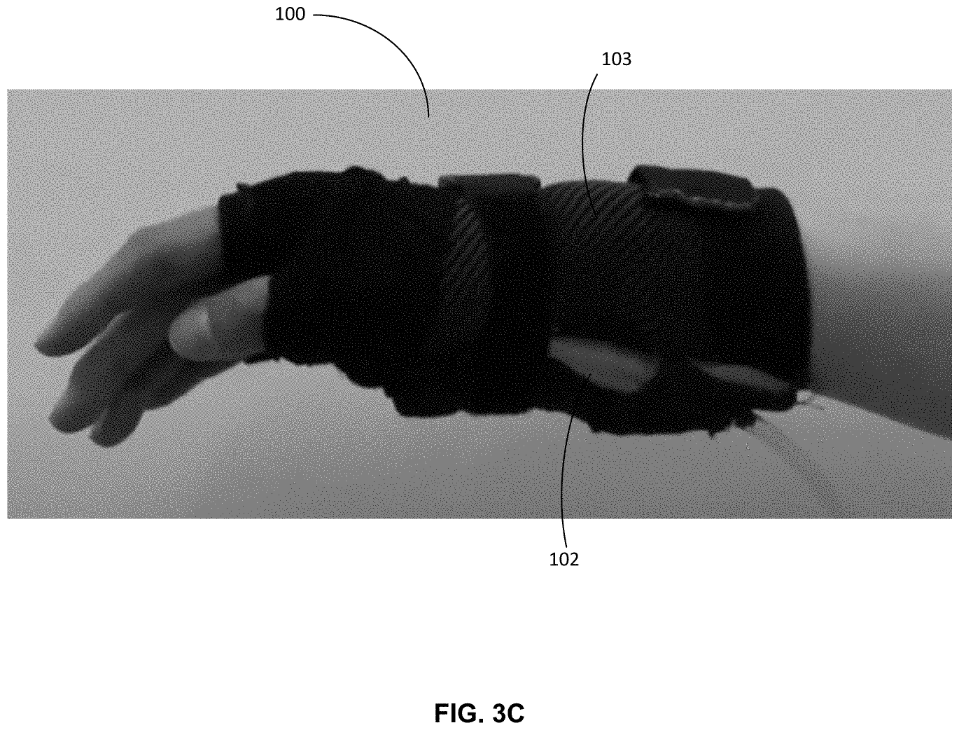

Components and principles of operation of system 100 are further illustrated in FIGS. 2, 3A, 3B, and 3C. While the principles of this disclosure have been shown in various embodiments, many modifications of structure, arrangements, proportions, the elements, materials and components, used in practice, which are particularly adapted for a specific environment and operating requirements may be used without departing from the principles and scope of this disclosure. These and other changes or modifications are intended to be included within the scope of the present disclosure.

The present disclosure has been described with reference to various embodiments. However, one of ordinary skill in the art appreciates that various modifications and changes can be made without departing from the scope of the present disclosure. Accordingly, the specification is to be regarded in an illustrative rather than a restrictive sense, and all such modifications are intended to be included within the scope of the present disclosure. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, required, or essential feature or element.

As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. Also, as used herein, the terms “coupled,” “coupling,” or any other variation thereof, are intended to cover a physical connection, an electrical connection, a magnetic connection, an optical connection, a communicative connection, a functional connection, and/or any other connection. When language similar to “at least one of A, B, or C” or “at least one of A, B, and C” is used in the specification or claims, the phrase is intended to mean any of the following: (1) at least one of A; (2) at least one of B; (3) at least one of C; (4) at least one of A and at least one of B; (5) at least one of B and at least one of C; (6) at least one of A and at least one of C; or (7) at least one of A, at least one of B, and at least one of C.

What is claimed is:

1. A compression sleeve wrist tremor stabilization system, comprising:
a first soft compliant actuator attached to a glove and disposed at the underside of the wrist; and
a second soft compliant actuator attached to the glove and disposed over the thenar eminence muscle group.

2. The system of claim 1, further comprising:
a microprocessor;
an accelerometer in electronic communication with the microprocessor; and
a compressor in electronic communication with the microprocessor and in pneumatic communication with the first soft compliant actuator and the second soft compliant actuator,
wherein, responsive to a detection of a wrist tremor via the accelerometer, the microprocessor transmits an instruction to the compressor to at least partially inflate at least one of the first soft compliant actuator or the second soft compliant actuator.

3. The system of claim 1, wherein the first soft compliant actuator is a pneunet.

4. The system of claim 2, further comprising a pressure transducer in electronic communication with the microprocessor, the pressure transducer configured to measure a force generated by inflation of at least one of the first soft compliant actuator or the second soft compliant actuator.

5. The system of claim 1, wherein the glove comprises a stretchable material.

5. The system of claim 1, wherein, when the first soft compliant actuator and the second soft compliant actuator are not inflated, the system does not impair the range of motion of the wrist of a wearer.

6. The system of claim 1, wherein the first soft compliant actuator and the second soft compliant actuator are removably attached to the glove.

7. The system of claim 1, wherein the first soft compliant actuator comprises silicone rubber.

8. The system of claim 1, wherein the first soft actuator comprises a thermoplastic polyurethane bag and a fabric cover.

9. The system of claim 2, further comprising a wireless transducer configured to transmit operational information regarding the system to a remote computer system.

10. A method of stabilizing an ataxia tremor, the method comprising:
detecting, via an accelerometer, an ataxia tremor; and
inflating, by a compressor, at least one of a first soft compliant actuator attached to a glove and disposed at the underside of the wrist or a second soft compliant actuator attached to the glove disposed over the thenar eminence muscle group.

11. The method of claim 10, further comprising:
detecting, via the accelerometer, cessation of the ataxia tremor; and
deflating, by the compressor, at least one of the first soft compliant actuator or the second soft compliant actuator.

12. The method of claim 10, further comprising:
deflating, by the compressor and responsive to expiration of a timer activated by the inflating, by the compressor, at least one of the first soft compliant actuator or the second soft compliant actuator.

13. The method of claim 10, further comprising:
detecting, by a pressure transducer configured to measure a force generated by at least one of the first soft compliant actuator or the second soft compliant actuator, a pressure threshold; and
responsive to the detecting, stopping inflation of at least one of the first soft compliant actuator or the second soft compliant actuator.

14. The method of claim 10, wherein the glove comprises a stretchable material.

15. The method of claim 10, wherein, when the first soft compliant actuator and the second soft compliant actuator are not inflated, the first soft compliant actuator and the second soft compliant actuator do not impair the range of motion of the wrist of a wearer.

16. The method of claim 10, wherein the first soft compliant actuator and the second soft compliant actuator are removably attached to the glove.
